(19) United States
(12) Patent Application Publication
(10) Pub. No.: US 2011/0065748 A1
(43) Pub. Date: Mar. 17, 2011

(54) COMPOSITIONS AND METHODS FOR TREATING DISORDERS OF GASTROINTESTINAL MOTILITY

(75) Inventor: John E. Donello, Dana Point, CA (US)

(73) Assignee: Allergan, Inc.

(21) Appl. No.: 12/882,965

(22) Filed: Sep. 15, 2010

Related U.S. Application Data
(60) Provisional application No. 61/243,060, filed on Sep. 16, 2009.
(60) Provisional application No. 61/246,238, filed on Sep. 28, 2009.

(51) Int. Cl. A61K 31/4709; A61K 31/4439; A61K 31/4545; A61K 31/4535; A61K 31/4025; A61K 31/402; A61P 1/00; A61P 1/10; A61P 35/00; A61P 1/16; A61P 29/00; A61P 1/08; A61P 1/12; A61P 1/14; A61P 1/18; A61P 1/06; A61P 1/04; A61P 9/10

(52) U.S. Cl. 514/314; 514/343; 514/318; 514/326; 514/422; 514/423

(57) ABSTRACT

Disclosed are methods of treating disorders of gastrointestinal motility by administering to a patient in need of such treatment a compound having the following formula:

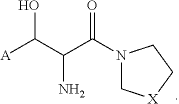

### CROSS REFERENCE

This application claims the benefit of U.S. Provisional Patent Application Ser. No. 61/243,060, filed on Sep. 16, 2009, and U.S. Provisional Patent Application Ser. No. 61/246,238, filed on Sep. 28, 2009, the entire disclosures of which are incorporated herein by this specific reference.

Disclosed herein is a method of treating disorders of gastrointestinal motility by administering to a patient in need of such treatment a compound having the following Formula I:

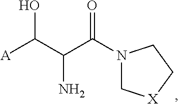

wherein X is CH₂ or CH₂—CH₂,
A is aryl, or is heteroaryl having 1, 2, or 3 atoms selected from the group consisting of N, S, and O,
wherein A has 0, 1, 2, or 3 substituents each comprising 0 to 8 carbon atoms, 0 to 3 oxygen atoms, 0 to 3 halogen atoms, 0 to 2 nitrogen atoms, 0 to 2 sulfur atoms, and 0 to 24 hydrogen atoms.

### DETAILED DESCRIPTION OF THE INVENTION

### Disorders of Gastrointestinal Motility

“Gastrointestinal motility” refers to the movement of food through the gastrointestinal tract. A “disorder of gastrointestinal motility” is any abnormality in that process that causes discomfort to a patient. It includes, for example, achalasia, Barrett's syndrome, biliary dyskinesia, Crohn's disease, chronic intestinal pseudo-obstruction, colonic inertia, constipation, cyclic vomiting syndrome, diarrhea, diffuse esophageal spasm, dumping syndrome, dyspepsia, dysphagia, encopresis, fecal incontinence, functional biliary disorders (e.g., functional biliary SO disorder, functional gallbladder disorder, functional pancreatic SO disorder, functional sphincter of Oddi disorder), functional bowel outlet obstruction, functional dyspepsia disorders (e.g., functional dyspepsia, postprandial distress syndrome), functional esophogeal disorders (e.g., functional dysphagia, functional heartburn, globus), functional fecal retention, gastroesophageal reflux disease (GERD), gastroparesis, gastritis, gastropathy, Hirschprung's disease, hypercontractile motility, hypermotility, hypertensive lower esophageal sphincter, hypomotility, intestinal obstruction, irritable bowel syndrome, ischemia, megacolon, non-erosive reflux disease, pancreatitis, pelvic floor dysfunction, short bowel syndrome, small bowel bacterial overgrowth, small bowel intestinal motility disorder, superior mesenteric artery syndrome, ulcerative colitis, and volvulus.

Compounds of the invention are useful for treating chronic pain, including pain associated with various disorders of gastrointestinal motility, but the inventors have discovered that the compounds may be used to treat such disorders themselves, as opposed to merely the pain associated with them. For example, in the treatment of gastrointestinal reflux, compounds of the invention may be used to inhibit transient lower esophageal sphincter relaxation. Hence, “disorders of gastrointestinal disorders” includes those symptoms, other than pain, produced by disorders of gastrointestinal motility that cause discomfort to a patient Disorder of gastrointestinal motility” also includes, therefore, altered bowel habit (including, for example, change in stool frequency; change in stool form, such as passing hard or loose stools; or change in the manner of passing stool, such as straining, urgency, or feeling or incomplete evacuation), belching, bloating (including a feeling of abdominal distension), blood or mucus in the stool, diarrhea, dyspepsia, dysphagia, flatulence, globus, hoarseness of voice, loss of appetite, nausea, (heartburn), regurgitation, sore throat, trapped gas, and uncomfortable fullness after meals.

### Compounds of the Invention

The method of the invention comprises administering to a patient compounds of Formula I:

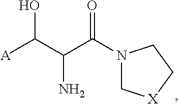

wherein X is CH₂ or CH₂—CH₂,
A is aryl, or is heteroaryl having 1, 2, or 3 atoms selected from the group consisting of N, S, and O, and
wherein A has 0, 1, 2, or 3 substituents each comprising 0 to 8 carbon atoms, 0 to 3 oxygen atoms, 0 to 3 halogen atoms, 0 to 2 nitrogen atoms, 0 to 2 sulfur atoms, and 0 to 24 hydrogen atoms.

“Aryl,” as used here, means any ring or ring system that contains at least one aromatic ring, such as phenyl, naphthyl, or biphenyl. Each ring may be substituted or unsubstituted.

“Heteroaryl,” as used here, means an aromatic ring or aromatic ring system in which 1, 2, or 3 of the atoms in at least one ring are N, S, or O. This includes, for example, monocyclic aryl rings wherein at least one nitrogen, oxygen, or sulfur atom is in the ring, and bicyclic aromatic ring systems wherein at least one nitrogen, oxygen, or sulfur atom is in at least one of the rings. Examples of heteroaryl include pyridinyl, furyl, thienyl, benzothienyl, benzofuryl, quinolinyl, imidazolyl, thiazolyl, oxazolyl, and the like. Each ring may be substituted or unsubstituted.

The substituents may be the same or different. Examples of substituents having the constraints defined here include, but are not limited to, the following:

hydrocarbyl, meaning a moiety consisting of carbon and hydrogen only, including, but not limited to,
a. alkyl, meaning hydrocarbyl having no double or triple bonds, including, but not limited to,
   i) linear alkyl, e.g. methyl, ethyl, n-propyl, n-butyl, n-pentyl, n-hexyl, etc.,
   ii) branched alkyl, e.g. iso-propyl, t-butyl and other branched butyl isomers, branched pentyl isomers, etc.,
   iii) cycloalkyl, e.g. cyclopropyl, cyclobutyl, cyclopentyl, cyclohexyl, etc., which may optionally be fused to another cycloalkyl or phenyl substituent;
   iv) combinations of linear, branched, and/or cycloalkyl;
b. alkenyl, e.g. hydrocarbyl having 1 or more double bonds, including linear, branched, or cycloalkenyl;
c. alkynyl, e.g. hydrocarbyl having 1 or more triple bonds, including linear or branched (alkynyl);

d. combinations of alkyl, alkenyl, and/or akynyl; alkyl-CN, such as —CH₂—CN, —(CH₂)₂—CN; —(CH₂)₃—CN, and the like;

hydroxyalkyl, i.e., alkyl-OH, such as hydroxymethyl, hydroxyethyl, and the like;

ether substituents, including —O-alkyl, alkyl-O-alkyl, and the like;

hydroxy alkyl ether, such as —COOH,

thioalkyl and thioether substituents, including —S-alkyl, alkyl-5-alkyl, and the like;

amine substituents, including —NH₂, —NH-alkyl, —N-alkyl¹alkyl² (i.e., alkyl¹ and alkyl² are the same or different, and both are attached to N), alkyl-NH₂, alkyl-NH-alkyl, alkyl-N-alkyl¹alkyl², and the like;

aminoalkyl, meaning alkyl-amine, such as aminomethyl (—CH₂-amine), aminoethyl, and the like;

ester substituents, including —CO₂-alkyl, —CO₂-phenyl, etc.;

other carbonyl substituents, including aldehydes; ketones, such as acyl (i.e.

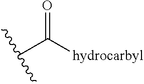

), and the like; in particular, acetyl, propionyl, and benzoyl substituents are contemplated;

phenyl and substituted phenyl; the phenyl and substituted phenyl may itself be optionally fused with another phenyl or cycloalkyl substituent;

fluorocarbons and hydrofluorocarbons such as —CF₃, —CH₂CF₃, etc.;

—CN; and

—F, —Cl, —Br, or —I.

Combinations of the foregoing substituents are also possible, subject to the constraints defined.

Substituents must be sufficiently stable to be stored in a bottle at room temperature under a normal atmosphere for at least 12 hours, or stable enough to be useful for any purpose disclosed herein.

If a substituent is a salt, for example of a carboxylic acid or an amine, the counter-ion of said salt, i.e. the ion that is not covalently bonded to the remainder of the molecule is not counted for the purposes of the number of heavy atoms in a substituent. Thus, for example, the salt —CO₂⁻Na⁺ is a stable substituent consisting of 3 heavy atoms, i.e. sodium is not counted. In another example, the salt —NH(Me)₂⁺Cl⁻ is a stable substituent consisting of 3 heavy atoms, i.e. chlorine is not counted.

In one embodiment, A is pyridinyl, meaning that compounds of structures such as those shown below are contemplated. In these structures, R1, R2, and R3 are substituents as defined herein:

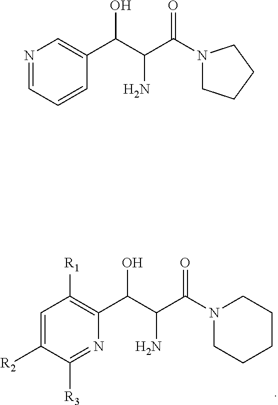

In another embodiment, A is thienyl, meaning that compounds of structures such as those shown below are contemplated. In these structures, R1 and R2 are substituents as defined herein:

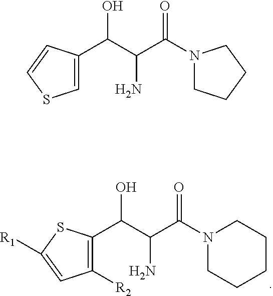

In another embodiment, A is furyl, meaning that compounds of structures such as those shown below are contemplated. In these structures, R1, R2, and R3 are substituents as defined herein:

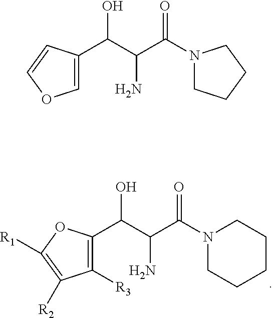

In one embodiment, each substituent is independently alkyl having from 1 to 8 carbon atoms.

In another embodiment, A is unsubstituted or has an isopropyl substituent.

In another embodiment, each substituent of A is —F, —Cl, —CH₃, or —CF₃.

In another embodiment, A is pyridyl, thienyl, furyl, pyrrolyl, pyrrolidinyl, imidazolyl, oxazolyl, thiazolyl, pyrazolyl, pyrimidinyl, quinolinyl, or pyrazinyl having 0, 1, 2, or 3 substituents.

Unless otherwise indicated, reference to a compound includes pharmaceutically acceptable salts, prodrugs, tautomers, alternate solid forms, and non-covalent complexes of a chemical entity of the depicted structure or chemical name.

A pharmaceutically acceptable salt is any salt of the parent compound that is suitable for administration to an animal or human. A pharmaceutically acceptable salt also refers to any salt which may form in vivo as a result of administration of an acid, another salt, or a prodrug which is converted into an acid or salt. A salt comprises one or more ionic forms of the compound, such as a conjugate acid or base, associated with one or more corresponding counter-ions. Salts can form from or incorporate one or more deprotonated acidic groups (e.g. carboxylic acids), one or more protonated basic groups (e.g. amines), or both (e.g. zwitterions).

Pharmaceutically acceptable salts of acidic functional groups may be derived from organic or inorganic bases. The salt may comprise a mono or polyvalent ion. Of particular interest are the inorganic ions, lithium, sodium, potassium, calcium, and magnesium. Organic salts may be made with amines, particularly ammonium salts such as mono, di and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

Tautomers are isomers that are in rapid equilibrium with one another. They often, but do not necessarily, include a transfer of a proton, hydrogen atom, or hydride ion. For example, the structures herein are intended to include, but are not limited to, the tautomeric forms shown below:

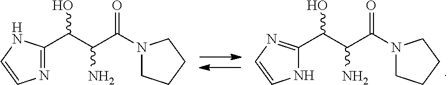

Unless stereochemistry is explicitly depicted, a structure includes every possible stereoisomer, both pure or in any possible mixture.

Alternate solid forms are different solid forms than ones that may result from practicing the procedures described herein. For example, alternate solid forms may be polymorphs, different kinds of amorphous solid forms, glasses, and the like.

Non-covalent complexes are complexes that may form between the compound and one or more additional chemical species that do not involve a covalent bonding interaction between the compound and the additional chemical species. They may or may not have a specific ratio between the compound and the additional chemical species. Examples might include solvates, hydrates, charge transfer complexes, and the like.

Methods for producing the compounds of the invention are described in, for example, U.S. Patent Application Publication No. 2009/0036436, the disclosure of which is incorporated herein by reference.

Compositions useful in the method of the invention may further include an excipient. Such an excipient may be a carrier or a diluent; this is usually mixed with the active compound, or permitted to dilute or enclose the active compound. If a diluent, the carrier may be solid, semi-solid, or liquid material that acts as an excipient or vehicle for the active compound. The formulations may also include wetting agents, emulsifying agents, preserving agents, sweetening agents, and/or flavoring agents.

Examples of compounds of the invention include the following

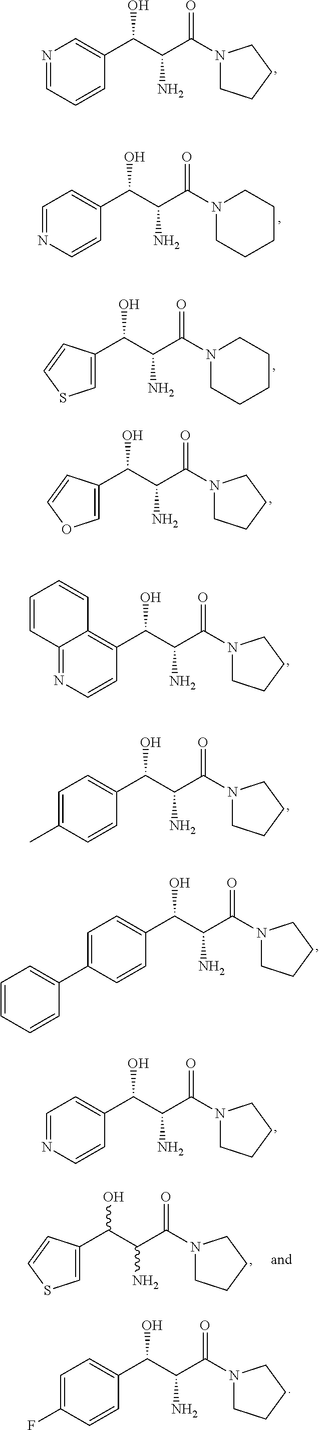

### Methods of Treatment

The compounds described here may be used to treat a patient suffering from disorders of gastrointestinal motility.

To “treat,” as used here, means to deal with medically. It includes, for example, administering a compound of the invention to prevent the onset of disorders of gastrointestinal motility, to alleviate their severity, and to prevent their reoccurrence.

The compounds of the invention may be administered at pharmaceutically effective amounts. Such amounts are normally the minimum dose necessary to achieve the desired therapeutic effect; in the treatment of disorders of gastrointestinal motility, this amount would be roughly that necessary to reduce the frequency and/or severity of the symptoms to tolerable levels. For human adults, pharmaceutically effective amounts will generally be in the range of 1-1,000 mg/day, including 1-25 mg/day, 25-50 mg/day, 50-75 mg/day, 75-100 mg/day, 100-150 mg/day, 150-200 mg/day, 200-250 mg/day, 250-300 mg/day, 300-350 mg/day, 350-400 mg/day, 400-450 mg/day, 450-500 mg/day, 500-550 mg/day, 550-600 mg/day, 600-650 mg/day, 650-700 mg/day, 700-750 mg/day, 750-800 mg/day, 800-850 mg/day, 850-900 mg/day, 900-950 mg/day, 950-1,000 mg/day. Higher doses (1,000-3,000 mg/day) may also be effective. The actual amount of the compound to be administered in any given case will be determined by a physician taking into account the relevant circumstances, such as the severity of the gastrointestinal motility disorder, the age and weight of the patient, the patient's general physical condition, and the route of administration. In one embodiment, the compounds of the invention are administered at doses that are pharmaceutically effective but that do not cause sedation.

The patient may be given the compounds of the invention orally in any acceptable form, such as a tablet, liquid, capsule, powder, and the like. Other routes may be desirable or necessary, particularly if the patient suffers from nausea. Such other routes may include, for example, transdermal, intraperitoneal, parenteral, subcutaneous, intranasal, intrathecal, intramuscular, intravenous and intrarectal modes of delivery.

What is claimed is:

1. A method for treating disorders of gastrointestinal motility, the method comprising the step of administering to a patient in need of such treatment a compound having the following structure: 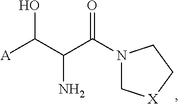
wherein X is CH₂ or CH₂—CH₂,
A is aryl, or is heteroaryl having 1, 2, or 3 atoms selected from the group consisting of N, S, and O, and
wherein A has 0, 1, 2, or 3 substituents each comprising 0 to 8 carbon atoms, 0 to 3 oxygen atoms, 0 to 3 halogen atoms, 0 to 2 nitrogen atoms, 0 to 2 sulfur atoms, and 0 to 24 hydrogen atoms.

2. The method of claim 1, wherein A is selected from the group consisting of pyridinyl, thienyl, furyl, quinolinyl, methylphenyl, and biphenyl.

3. The method of claim 2, wherein A is unsubstituted.

4. The method of claim 2, wherein the disorder is selected from the group consisting of achalasia, Barrett's syndrome, biliary dyskinesia, Crohn's disease, chronic intestinal pseudo-obstruction, colonic inertia, constipation, cyclic vomiting syndrome, diarrhea, diffuse esophageal spasm, dumping syndrome, dyspepsia, dysphagia, encopresis, fecal incontinence, functional biliary disorders (e.g., functional biliary SO disorder, functional gallbladder disorder, functional pancreatic SO disorder, functional sphincter of Oddi disorder), functional bowel outlet obstruction, functional dyspepsia disorders (e.g., functional dyspepsia, postprandial distress syndrome), functional esophogeal disorders (e.g., functional dysphagia, functional heartburn, globus), functional fecal retention, gastroesophageal reflux disease (GERD), gastroparesis, gastritis, gastropathy, Hirschprung's disease, hypercontractile motility, hypermotility, hypertensive lower esophageal sphincter, hypomotility, intestinal obstruction, irritable bowel syndrome, ischemia, megacolon, non-erosive reflux disease, pancreatitis, pelvic floor dysfunction, short bowel syndrome, small bowel bacterial overgrowth, small bowel intestinal motility disorder, superior mesenteric artery syndrome, ulcerative colitis, and volvulus.

5. The method of claim 1, wherein the compound is selected from the group consisting of

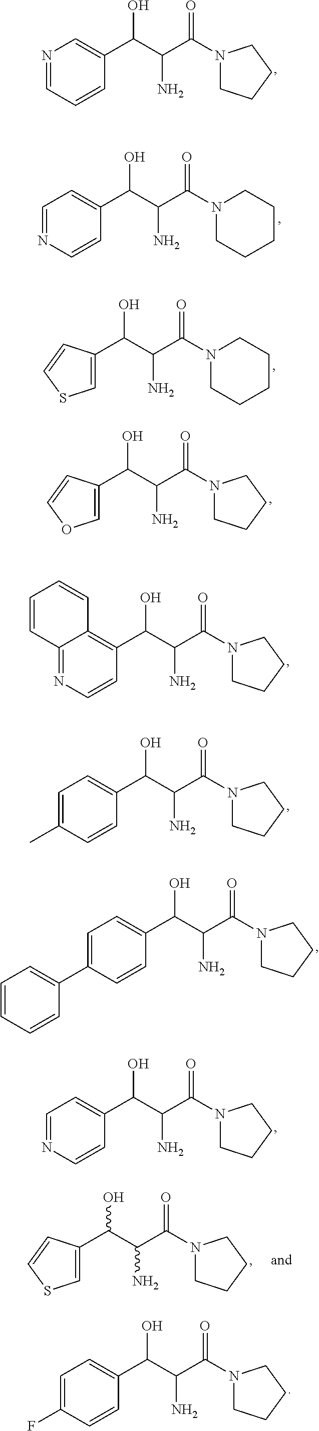

6. A method of inhibiting transient lower esophageal sphincter relaxation, the method comprising the step of administering to a patient in need of such inhibition a compound having the following structure: 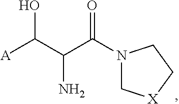
wherein X is CH₂ or CH₂—CH₂,
A is aryl, or is heteroaryl having 1, 2, or 3 atoms selected from the group consisting of N, S, and O, and
wherein A has 0, 1, 2, or 3 substituents each comprising 0 to 8 carbon atoms, 0 to 3 oxygen atoms, 0 to 3 halogen atoms, 0 to 2 nitrogen atoms, 0 to 2 sulfur atoms, and 0 to 24 hydrogen atoms.

7. The method of claim 6, wherein A is selected from the group consisting of pyridinyl, thienyl, furyl, quinolinyl, methylphenyl, and biphenyl.

8. The method of claim 7, wherein A is unsubstituted.

9. The method of any of claim 6, wherein the compound is selected from the group consisting of [chemical structure]
